(19) United States
(12) Patent Application Publication
(10) Pub. No.: US 2026/0367056 A1
(43) Pub. Date: Nov. 19, 2026

(54) MONITORING DEVICE FOR AN AGRICULTURAL FARM

(71) Applicants: S.C.R. (ENGINEERS) LIMITED, Netanya (IL); PTS TECHNOLOGIES PTE LIMITED, Singapore (SG)

(72) Inventors: Alan Huw Beynon, Devon (GB); John Keith Evans (deceased), Gwynedd (GB); Kim Huat Albert Loh, Singapore (SG); Lwin Aye Han, Singapore (SG)

(73) Assignees: S.C.R. (ENGINEERS) LIMITED, Netanya (IL); PTS TECHNOLOGIES PTE LIMITED, Singapore (SG)

(21) Appl. No.: 19/103,248

(22) PCT Filed: Aug. 15, 2023
(86) PCT No.: PCT/SG2023/050557

§ 371 Date: Feb. 12, 2025

(30) Foreign Application Priority Data
Aug. 16, 2022 (EP) 22190628.2

(51) Int. Cl. G01N 33/00; A01G 9/24; G01J 1/42

(52) U.S. Cl. CPC G01N 33/0068; A01G 9/24; G01J 1/4204; G01N 33/004

(57) ABSTRACT

The present disclosure generally relates to a monitoring device (100) for monitoring an environment of an agricultural farm. The monitoring device (100) comprises sensors (120) inside a housing body (110) for measuring conditions of the environment including temperature and humidity; ports (130) on the housing body (110) that are sealed with hydrophobic membranes (140) that prevent liquid and particulate ingress while allowing air communication through to the sensors (120); and a cover member (150) over the ports (130) to protect the sensors (120) from animal attacks, wherein the cover member (150) is spaced apart from the housing body (110) to facilitate air communication to the sensors (120).

### TECHNICAL FIELD

The present disclosure generally relates to a monitoring device for an agricultural farm. More particularly, the present disclosure describes various embodiments of a monitoring device for monitoring an environment of the agricultural farm.

### BACKGROUND

The National Geographic Society defines agriculture as the art and science of cultivating the soil, growing crops, and raising livestock. Crops are grown and livestock are raised in agriculture settings or farms for various purposes including producing food for human consumption. Environmental conditions such as temperature and humidity can affect agriculture productivity. For example, temperatures and/or humidity levels that are too high or too low can cause discomfort to livestock and/or increase the risk of diseases which would in turn reduce productivity. Imbalances in temperatures and/or humidity levels can cause detrimental effects on plants and potentially cause entire crop harvests to be wasted. Hence, agricultural farms often have thermometers and hygrometers to measure temperature and humidity and check that they are within acceptable levels. However, the thermometers and hygrometers are installed in agricultural farms and they would be exposed to harsh weather conditions such as heavy rainfall or intense sunlight. The sensitive electronic parts in the thermometers and hygrometers can get damaged for example due to water ingress from rain. Moreover, the thermometers and hygrometers are vulnerable to attacks by the livestock, birds, rodents, etc. and the sensitive electronic parts can get damaged.

Therefore, in order to address or alleviate at least one of the aforementioned problems and/or disadvantages, there is a need to provide an improved monitoring device for monitoring an environment of an agricultural farm.

### SUMMARY

According to an aspect of the present disclosure, there is a monitoring device for monitoring an environment of an agricultural farm. The monitoring device comprises:
a housing body;
a set of sensors inside the housing body for measuring conditions of the environment, the sensors comprising a temperature sensor and a humidity sensor for measuring temperature and humidity in the environment;
a set of ports on the housing body for exposing the sensors to the environment for measuring the environmental conditions;
a set of hydrophobic membranes sealing the ports to prevent liquid and particulate ingress while allowing air to communicate through the hydrophobic membranes to the sensors, such that the temperature and humidity sensors can measure the temperature and humidity of the air; and
a cover member couplable to the housing body over the ports for protecting the sensors from animal attacks, the cover member spaced apart from the housing body to facilitate communication of the air through the hydrophobic membranes to the sensors.

Devices, systems, and methods for monitoring an environment of an agricultural farm according to the present disclosure is thus disclosed herein. Various features, aspects, and advantages of the present disclosure will become more apparent from the following detailed description of the embodiments of the present disclosure, by way of non-limiting examples only, along with the accompanying drawings.

### BRIEF DESCRIPTION OF THE DRAWINGS

FIGS. 1A and 1B are illustrations of the monitoring device according to first embodiments of the present disclosure.

FIGS. 2A to 2C are illustrations of the monitoring device according to second embodiments of the present disclosure.

FIG. 3 is a block diagram of a monitoring system comprising a set of the monitoring devices and a computer system.

### DETAILED DESCRIPTION

For purposes of brevity and clarity, descriptions of embodiments of the present disclosure are directed to a monitoring device for monitoring an environment of an agricultural farm, in accordance with the drawings. While aspects of the present disclosure will be described in conjunction with the embodiments provided herein, it will be understood that they are not intended to limit the present disclosure to these embodiments. On the contrary, the present disclosure is intended to cover alternatives, modifications and equivalents to the embodiments described herein, which are included within the scope of the present disclosure as defined by the appended claims. Furthermore, in the following detailed description, specific details are set forth in order to provide a thorough understanding of the present disclosure. However, it will be recognised by an individual having ordinary skill in the art, i.e. a skilled person, that the present disclosure may be practiced without specific details, and/or with multiple details arising from combinations of aspects of particular embodiments. In a number of instances, well-known systems, methods, procedures, and components have not been described in detail so as to not unnecessarily obscure aspects of the embodiments of the present disclosure.

In embodiments of the present disclosure, depiction of a given element or consideration or use of a particular element number in a particular figure or a reference thereto in corresponding descriptive material can encompass the same, an equivalent, or an analogous element or element number identified in another figure or descriptive material associated therewith.

References to “an embodiment/example”, “another embodiment/example”, “some embodiments/examples”, “some other embodiments/examples”, and so on, indicate that the embodiment(s)/example(s) so described may include a particular feature, structure, characteristic, property, element, or limitation, but that not every embodiment/example necessarily includes that particular feature, structure, characteristic, property, element or limitation. Furthermore, repeated use of the phrase “in an embodiment/example” or “in another embodiment/example” does not necessarily refer to the same embodiment/example.

The terms “comprising”, “including”, “having”, and the like do not exclude the presence of other features/elements/steps than those listed in an embodiment. Recitation of certain features/elements/steps in mutually different embodiments does not indicate that a combination of these features/elements/steps cannot be used in an embodiment.

As used herein, the terms “a” and “an” are defined as one or more than one. The use of “/” in a figure or associated text is understood to mean “and/or” unless otherwise indicated. The term “set” is defined as a non-empty finite organisation of elements that mathematically exhibits a cardinality of at least one (e.g. a set as defined herein can correspond to a unit, singlet, or single-element set, or a multiple-element set), in accordance with known mathematical definitions.

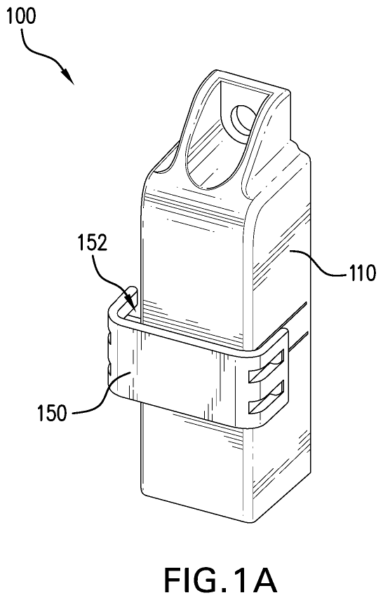

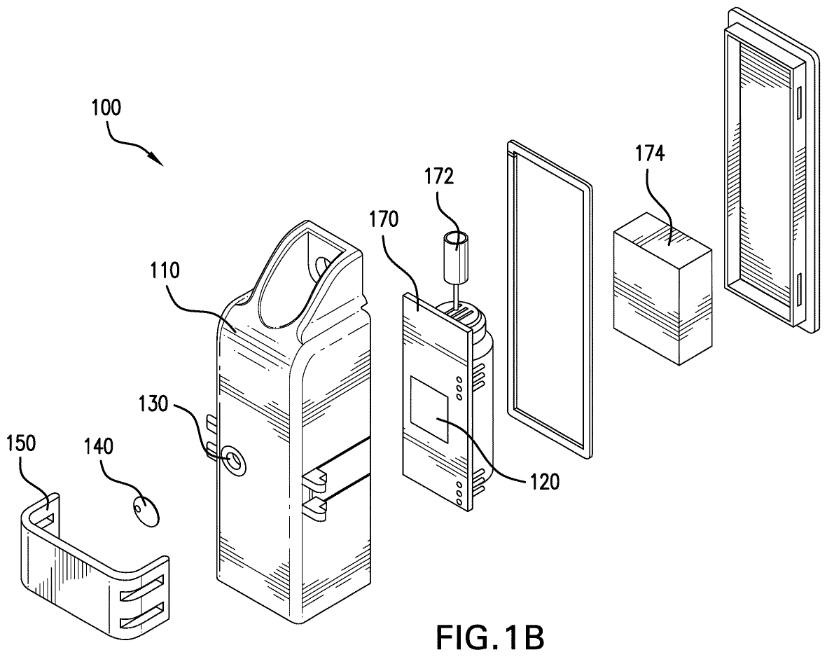

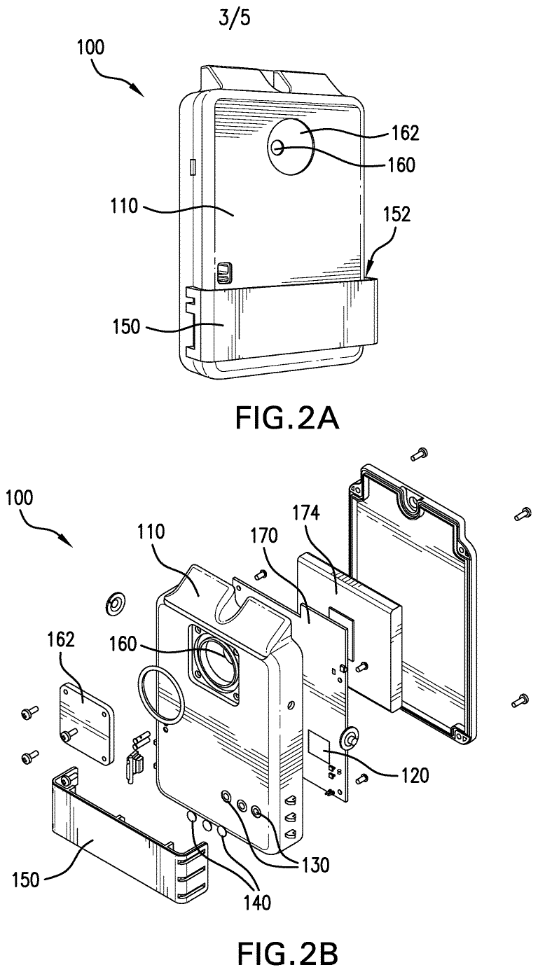

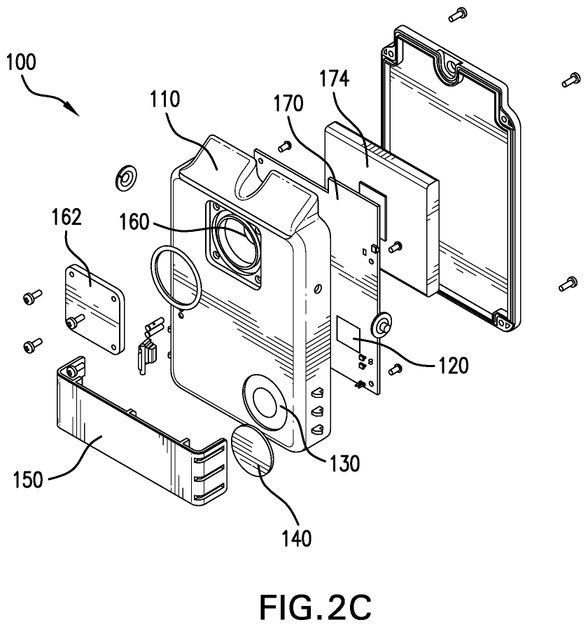

In representative or exemplary embodiments of the present disclosure, there is a monitoring device 100 for monitoring an environment of an agricultural farm. The agricultural farm may be one that raises livestock animals, such as cattle, goats, swine, and/or poultry, to produce food such as meat, milk, and eggs. Alternatively, the agricultural farm may be one that grows plants and cultivates food crops. Further alternatively, the agricultural farm may be one that produces both livestock and food crops. FIGS. 1A and 1B illustrate first embodiments of the monitoring device 100 while FIGS. 2A to 2C illustrate second embodiments of the monitoring device 100.

As shown in FIGS. 1A to 2C, the monitoring device 100 includes a housing body 110 for housing various components of the monitoring device 100 therein. The monitoring device 100 includes a set of one or more sensors 120 inside the housing body 110 for measuring conditions of the environment. In this example, the sensors 120 include a temperature sensor and a humidity sensor for measuring temperature and humidity in the environment. The monitoring device 100 includes a set of ports 130 on the housing body 110 for exposing the sensors 120 to the environment for measuring the environmental conditions. The monitoring device 100 includes a set of hydrophobic membranes 140 sealing the ports 130 to prevent liquid and particulate ingress while allowing air to communicate through the hydrophobic membranes 140 to the sensors 120, such that the temperature and humidity sensors can measure the temperature and humidity of the air.

Humidity refers to the amount of water vapour in the air and the humidity sensor and the humidity sensor and/or hygrometer measures this parameter. In many embodiments, the humidity sensor is configured to measure the relative humidity, which is the amount of water vapour in the air compared to the maximum possible amount at that temperature as measured by the temperature sensor or thermometer. In one example, the accuracy of the humidity sensor is about of +2% for an operating range of 20% RH to 80% RH. In one example sensor, the measurement range of the temperature sensor may be from 0 °C to 80 °C with an accuracy of about ±0.2 C.

In order for the humidity sensor to measure the water vapour in the air, water content must reach the humidity sensor housed inside the housing body 110. Existing hygrometers are openly exposed to ambient air so that they can reliably measure the water vapour in the air. However, excessive water content, especially during rainy conditions or even snowfall, can cause water damage to the existing hygrometers and nearby electronic components, potentially ending their useful life spans prematurely. The monitoring device 100 provides the hydrophobic membranes 140 to address this problem. The hydrophobic membrane 140 is a porous semi-permeable membrane that is structured to allow moist air, i.e. air and water vapour, to permeate through. Additionally, the hydrophobic membrane 140 repels liquid water as well as other liquids, thereby filtering out liquids and solid particles and preventing them from permeating through. Hence, the hydrophobic membranes 140 prevent liquid and particulate ingress into the ports 130 and to the sensors 120 inside the housing body 110, thereby preventing the sensors 120 and other electronic components inside the housing body 110 from getting damaged by liquid and particulate ingress. At the same time, the hydrophobic membranes 140 enable moist air to flow through to the sensors 120 so that the temperature and humidity of the air can still be reliably measured.

The housing body 110 together with the ports 130 sealed with the hydrophobic membranes 140 seal the sensors 120 and other electronic components inside the housing body 110, protecting them from liquid and particulate ingress. Preferably, the housing body 110 has an ingress protection rating of at least IP65, which means protection from total dust ingress and water jets from any direction.

In an example such as shown in FIG. 2C, one sensor 120 is paired with one port 130 and one hydrophobic membrane 140 sealing the port 130. In an example, one sensor is paired with plural ports 130 and each port 130 is sealed with a respective hydrophobic membrane 140. In an example, one sensor is paired with plural ports 130 and the plural ports 130 are sealed with a common hydrophobic membrane 140. In an example, there are plural sensors 120 and each sensor 120 is paired with a respective port 130 and the plural sensors 120 are paired with a respective hydrophobic membrane 140 (such as shown in FIG. 2B) or a common hydrophobic membrane 140. In an example, the plural sensors 120 are paired with a common port 130 and a common hydrophobic membrane 140. While some non-limiting examples have been described above, it will be appreciated that the monitoring device 100 can have various combinations and arrangements of the sensors 120, ports 130, and hydrophobic membranes 140, depending on the needs of the monitoring device 100.

The monitoring device 100 further includes a cover member 150 couplable to the housing body 110 over the ports 130 for protecting the sensors 120 and the hydrophobic membranes 140 from damage, such as due to animal attacks, rough handling of the monitoring device 100, sharp objects, harsh weather, or environmental conditions, etc. The cover member 150 may be integrally formed with or coupled to the housing body 110 or removably couplable to the housing body 110. The cover member 150 is spaced apart from the housing body 110 (forming a gap 152) to facilitate communication of the air through the hydrophobic membranes 140 to the sensors 120.

When the monitoring device 100 is installed in an agricultural farm that raises livestock animals such as cattle, swine, goats, or poultry, the monitoring device 100 is preferably placed at the height level of the livestock animals to obtain accurate measurements of the environmental conditions that the livestock animals are exposed to. For example, the monitoring device 100 may be placed at a height level (relative to the ground) ranging from around 10 cm to around 100 cm or more, depending on the height of the livestock animals which would be different for cattle, swine, goats, or poultry. For an agricultural farm raising poultry, the monitoring device 100 can be placed at a height level of around 20 cm.

As the monitoring device 100 is placed at the height level of the animals, some animals may try to attack the monitoring device 100 depending on how aggressive the animals are to foreign objects in their environment. For example, swine or pigs tend to eat objects more aggressively than other livestock animals. Moreover, depending on the height level, the monitoring device 100 may attract flight animals like birds to fly towards and attack the monitoring device 100. The cover member 150 thus shields the ports 130 and underlying sensors 120 from attacks by the livestock animals, as well as from birds which might otherwise peck at the ports 130 and damage the hydrophobic membranes 140 and/or sensors 120 if the cover member 150 was removed or damaged.

It will be appreciated that the monitoring device 100 may be modified according to design needs such as based upon the type of livestock animals and/or the environment of the agricultural farm. For example, the housing body 110 may be made larger if the animals are larger and/or more aggressive to foreign objects, so that the monitoring device 100 is less vulnerable to damage from attacks by these animals. The housing body 110 can be designed with various colours depending on design needs. More particularly, it was found that if the housing body 110 is white, birds were less likely to interfere with and attack the monitoring device 100, and the risk of damage is lower.

Further, as the monitoring device 100 is placed relatively close to the ground, it might be more exposed to rough conditions in the farm, such as when the farm is being cleaned. For example, harsh cleaning equipment such as water jets and chemical agents may be used to clean the farm. The cover member 150 and hydrophobic membranes 140 can protect the sensors 120 and other internal components inside the housing body 110, particularly sensitive electronic components which can be corroded by chemical agents.

In the second embodiments as shown in FIGS. 2A to 2C, the sensors 120 may further include a barometric pressure sensor for measuring atmospheric pressure of the air. The measurement range of the barometric pressure sensor may be from 26 kPa to 126 kPa. The sensors 120 may further include one or more gas sensors for measuring an amount of one or more gases in the air. For example, the gas sensors can include a carbon dioxide (CO₂) gas sensor for measuring the amount of CO₂ in the air. The measurement range of the CO₂ gas sensor may be from around 400 ppm to around 10,000 ppm, or preferably from 400 ppm to around 4,000 ppm, with an accuracy of about ±3%.

In the second embodiments as shown in FIGS. 2A to 2C, the monitoring device 100 may further include an illumination sensor 160 inside the housing body 110 for measuring light intensity in the environment. The agricultural farm may provide various types of lighting, such as sunlight, LED light, fluorescent light, and specific coloured light, to help the growth animals and crops in the farm. The monitoring device 100 may further include a transparent member 162 on the housing body 110 and covering the illumination sensor 160 to protect the illumination sensor 160 from animal attacks. The transparent member 162 works to protect the illumination sensor 160 similarly to the cover member 150 protecting the sensors 120. Additionally, the transparent member 162 is optically transparent so that the illumination sensor 160 can reliably measure the light intensity of the lighting in the farm environment. The measurement range of the illumination sensor 160 may be from around 0.01 lux to around 83,000 lux.

It will be appreciated that the first and second embodiments described with reference to FIGS. 1A to 2C can include any combination of the sensors 120 (including the temperature sensor, humidity sensor, barometric pressure sensor, and gas sensor) and the illumination sensor 160. In these embodiments, the monitoring device 100 may further include a set of electronic components inside the housing body 110, the electronic components including a computer processor for processing data measured by the sensors 120 and illumination sensor 160 if available (collectively referred to as sensors 120,160). The data can thus include data about temperature, humidity, pressure, gaseous components (e.g. CO₂), and/or light intensity of the environment.

The electronic components may further include a wireless communications component for communicating the data to a remote computer system 200 for remotely monitoring the environment of the agricultural farm. The electronic components may further include an antenna 172 for boosting the wireless communications to the remote computer system 200. The electronic components and the sensors 120,160 may be integrated in a printed circuit board (PCB) 170.

The computer processor may be configured for controlling the sensors 120,160 and wireless communications component to perform measurements, for example at predefined intervals or for certain durations, and to transmit the data, for example at predefined intervals, continuously, or some combination. For example, the temperature sensor and humidity sensor may be controlled to perform measurements continuously and/or at predefined intervals (such as 5-minute, 10-minute, 15-minute, or 30-minute intervals), the CO₂ gas sensor may be controlled to perform measurements continuously and/or at predefined intervals (such as 5-second, 10-second, 15-second, 30-second intervals), and the wireless communications component may be controlled to transmit the data continuously and/or at predefined intervals (such as 5-minute, 10-minute, 15-minute, or 30-minute intervals). The computer processor may be configured to adjust the measurement and transmission intervals as desired, such as based on instructions received from the remote computer system 200.

The electronic components further include one or more power source 174, such as a rechargeable battery 174 for powering the electronic components and the sensors 120,160. In one embodiment, the power source 174 is a rechargeable battery that is connected to the PCB 170 and a full charge can last for about 40 days. Additionally, the housing body 110 housing the battery 174 also seals any chemical leakages from the battery 174 within the housing body 110, thereby protecting the agricultural farm from such chemical leakages.

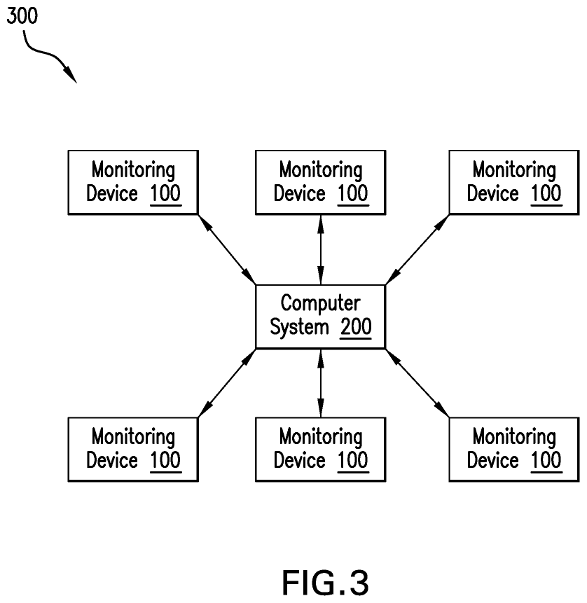

In representative or exemplary embodiments of the present disclosure, with reference to FIG. 3, there is a monitoring system 300 for monitoring the environment of the agricultural farm. The monitoring system 300 includes a set of monitoring devices 100 distributed in the agricultural farm. It will be appreciated that the monitoring devices 100 may be arranged at strategic locations in the agricultural farm to obtain a holistic understanding of the environmental conditions of the whole agricultural farm. The monitoring devices 100 may be arranged within a subsection of the farm, for example, within a specific barn or yard.

The monitoring system 300 may further include the remote computer system 200 communicative with the monitoring devices 100 for remotely monitoring the environment of the agricultural farm. More specifically, the remote computer system 200 includes a gateway server that manages secure gateway connections with and configurations of the network of monitoring devices 100. The remote computer system 200 and the wireless communications components of the monitoring devices 100 are configured for communicating with each other using a suitable wireless communication protocol.

In many embodiments, the communications between the monitoring devices 100 and the remote computer system 200 is based on the LoRa® wireless communication protocol which employs the LoRaWAN® specification. The LoRaWAN® specification is a Low Power, Wide Area (LPWA) networking protocol that enables data transmissions over long distances up to a few kilometres, making it suitable for use in agricultural farms that span across large areas of land.

To enable wireless communications between a monitoring device 100 and the remote computer system 200, the monitoring device 100 first needs to be paired with the remote computer system 200. The monitoring device 100 may be pre-programmed or predefined with a set of identification data or credentials, such as including a network address for use in communications within a network centred at the remote computer system 200. One example of the network address is a media access control (MAC) address. The LoRaWAN® specification describes certain device credentials that should be predefined for the monitoring device 100.

Additionally, the LoRaWAN® specification has a large network capacity for the network of monitoring devices 100, making it suitable for use with many monitoring devices 100 distributed across large areas which can be connected to a single remote computer system 200. This significantly reduces the costs of deploying multiple computer systems 200 and the manpower needed to operate and maintain them. Thus, use of the monitoring system 300 can help reduce operating costs, as well as increase productivity and revenue stream for the agricultural farm.

The remote computer system 200 may further include an online interface 240 for viewing the data. The online interface may include various functionalities such as data analytics to analyse the data to check that they are within acceptable levels and evaluate the environmental conditions of the agricultural farm.

Therefore, monitoring the environment of the agricultural farm allows for early remedial actions to be taken when the environmental conditions exceed acceptable levels. This lowers the risk of damage to food crops and livestock animals that could have resulted from harsh environments. Moreover, for livestock animals, the monitoring device 100 is placed at the height level of the animals so that the environmental conditions are measured more accurately. A truer representation of the farm environment which the livestock animals are exposed to can be assessed and more effective remedial actions can be taken. The risk of diseases and their mortality rates can be reduced if the animals are kept comfortable in the agricultural farm. There is hence lower risk of the agricultural farm suffering from poorer productivity due to harsh environments which would have resulted in revenue losses.

Some non-limiting examples of the remote computer system 200 include computers, laptops, mini-computers, mainframe computers, any non-transient and tangible machines that can execute a machine-readable code, cloud-based servers, distributed server networks, and a network of computer systems. As used herein, a server is a physical or cloud data processing system on which a server program runs. The server may be implemented in hardware or software, or a combination thereof. Additionally, the remote computer system 200 includes a processor, a memory, and various other modules or components. The modules and components thereof are configured for performing various operations or steps and are configured as part of the processor. Such operations or steps are performed in response to non-transitory instructions operative or executed by the processor. The memory is used to store instructions and perhaps data which are read during program execution. The memory may be referred to in some contexts as computer-readable storage media and/or non-transitory computer-readable media. Non-transitory computer-readable media include all computer-readable media, with the sole exception being a transitory propagating signal per se.

In the foregoing detailed description, embodiments of the present disclosure in relation to a monitoring device for monitoring an environment of an agricultural farm is described with reference to the provided figures. The description of the various embodiments herein is not intended to call out or be limited only to specific or particular representations of the present disclosure, but merely to illustrate non-limiting examples of the present disclosure. The present disclosure serves to address at least one of the mentioned problems and issues associated with the prior art. Although only some embodiments of the present disclosure are disclosed herein, it will be apparent to a person having ordinary skill in the art in view of this disclosure that a variety of changes and/or modifications can be made to the disclosed embodiments without departing from the scope of the present disclosure. Therefore, the scope of the disclosure as well as the scope of the following claims is not limited to embodiments described herein.

1. A monitoring device for monitoring an environment of an agricultural farm, the monitoring device comprising:
a housing body;
a set of sensors inside the housing body for measuring conditions of the environment, the sensors comprising a temperature sensor and a humidity sensor for measuring temperature and humidity in the environment;
a set of ports on the housing body for exposing the sensors to the environment for measuring the environmental conditions;
a set of hydrophobic membranes sealing the ports to prevent liquid and particulate ingress while allowing air to communicate through the hydrophobic membranes to the sensors, such that the temperature and humidity sensors can measure the temperature and humidity of the air; and
a cover member couplable to the housing body over the ports for protecting the sensors from animal attacks, the cover member spaced apart from the housing body to facilitate communication of the air through the hydrophobic membranes to the sensors.

2. The monitoring device according to claim 1, wherein the sensors further comprise a barometric pressure sensor for measuring atmospheric pressure of the air.

3. The monitoring device according to claim 1, wherein the sensors further comprise one or more gas sensors for measuring an amount of one or more gases in the air.

4. The monitoring device according to claim 3, wherein the gases comprise carbon dioxide.

5. The monitoring device according to claim 1, further comprising:
an illumination sensor inside the housing body for measuring light intensity in the environment; and
a transparent member on the housing body and covering the illumination sensor to protect the illumination sensor from animal attacks.

6. The monitoring device according to claim 1, further comprising a set of electronic components inside the housing body, the electronic components comprising a computer processor for processing data measured by the sensors.

7. The monitoring device according to claim 6, wherein the electronic components further comprise a wireless communications component for communicating the data to a remote computer system for remotely monitoring the environment of the agricultural farm.

8. The monitoring device according to claim 7, wherein the wireless communications component is configured for communications using a LoRa® wireless communication protocol.

9. The monitoring device according to claim 7, wherein the computer processor is configured for controlling the wireless communications component to transmit the data to the remote computer system at predefined intervals.

10. The monitoring device according to claim 6, wherein the computer processor is configured for controlling the sensors to perform measurements at predefined intervals.

11. The monitoring device according to claim 1, wherein the housing body has an ingress protection rating of at least IP65.
